SEQUENCE LISTING (10 sequences)

SEQ ID NO: 1 - DNA, 20 bp, Bovine herpesvirus 1
gaccctcgccgatatttatt

SEQ ID NO: 2 - DNA, 20 bp, Bovine herpesvirus 1
gagggaccacagagaaggat

SEQ ID NO: 3 - DNA, 20 bp, Bovine herpesvirus 1
gagggaccacagagaaggat

SEQ ID NO: 4 - DNA, 20 bp, Bovine herpesvirus 1
gaccctcgccgatatttatt

SEQ ID NO: 5 - DNA, 20 bp, Bovine herpesvirus 1
gcgtcgtttctaaagagcag

SEQ ID NO: 6 - DNA, 20 bp, Bovine herpesvirus 1
gagctgttcacccaaaaaga

SEQ ID NO: 7 - DNA, 20 bp, Bovine herpesvirus 1
gccgcaagtttatgctgtat

SEQ ID NO: 8 - DNA, 20 bp, Bovine herpesvirus 1
catcgaggcagtgtaggtct

SEQ ID NO: 9 - DNA, 20 bp, Bovine herpesvirus 1
ggtgcattgagcttgacttt

SEQ ID NO: 10 - DNA, 20 bp, Bovine herpesvirus 1
gtacttgttggggacacagg

(19) United States
(12) Patent Application Publication
(10) Pub. No.: US 2010/0047308 A1
(43) Pub. Date: Feb. 25, 2010

(54) Production methods of virus inactivated and cell-free body implant

(76) Inventors: Jin Young Kim, Chungcheongnam-do (KR); In Seop Kim, Daejeon (KR); Jae Hyoung Ahn, Daejeon (KR); Hyo Sun Jeong, Daejeon (KR); Seok Beam Song, Daejeon (KR); Ji Hwa Chae, Daejeon (KR); Seog Jin Seo, Daejeon (KR); Ke Won Kang, Daejeon (KR); Ho Chan Hwang, Seoul (KR)

Correspondence Address: CLARK & ELBING LLP, 101 FEDERAL STREET, BOSTON, MA, 02110, US

(21) Appl. No.: 12/311,398

(22) PCT Filed: Sep. 28, 2007
(86) PCT No.: PCT/KR2007/004750

§ 371 Date: Mar. 27, 2009

(30) Foreign Application Priority Data
Sep. 26, 2006 (KR) 10-2006-095322

(51) Int. Cl. A61K 35/36; A61F 2/02; A01N 57/10; A01P 1/00; A61P 17/02

(52) U.S. Cl. 424/423; 623/11.11; 514/144; 424/93.7

(57) ABSTRACT

A method is provided for producing a virus-inactivated, acellular implant for the human body, featuring the use of TNBP in combination with a detergent selected from among deoxycholate, SDS, Tween 80, Triton X-100, sodium cholate and combinations thereof for removing cells and viruses simultaneously. Also disclosed are an acellular human body implant produced by the method and a wound healing agent comprising the acellular human body implant.

### TECHNICAL FIELD

The present invention relates, in general, to a method for producing an implant for the human body using a solvent and a detergent in combination and, more particularly, to a method for producing an acellular dermal implant for the human body by removing cells and viruses simultaneously through the use of a solvent in combination with a detergent.

### BACKGROUND ART

The skin is largely divided into epidermal tissue, accounting for the outer layer of the skin, and a dermis layer located just below the epidermis. The epidermis primarily functions as a protective barrier against moisture loss in the body and against external harmful substances, such as pathogens, UV light, chemicals, etc. Keratinocytes makes up the outermost layer of the epidermis while there are also present various components including melanocytes, responsible for blocking UV radiation, Langerhans cells, responsible for dermal immunity, follicular cells, responsible for hair growth, and sweat glands. Basal cells are located in the innermost layer of the epidermis. Basal cells, although having no protective functions, serve as a source of various cells on the protective frontline. Wound healing is accomplished through the generation of new cells from the basal cells. The epidermis contains no blood vessels, and cells in the deepest layers are nourished by diffusion from blood capillaries extending to the upper layers of the dermis, which consists mainly of fibrous proteins (collagen) with fibroblasts studded therein (Weekly DongA, Vol. 322, Feb. 14, 2002).

An acellular dermal implant is designed to reconstruct the skin in patients with skin defect, such as burns, wounds from traffic accidents, ulcers, etc. In addition, the acellular dermal implant is applicable to various loci of the human body, including the nasal septum as well as all skin layers, irrespective of human race, sex, and age, in order to reconstruct injured skin, such as, e.g., reconstruction of injured dura mater, correction of depressed scars, correction of hemifacial microsomia, plastic reconstruction for lip enlargement, etc.

Because the biological properties thereof are different from those of general medicines, however, skin implants have long been disputed with regard to the activation of endogenous and exogenous contaminants or the contamination of pathogens.

Before use, typically, blood-derived medicines undergo virus clearance processes, such as heat inactivation, treatment with solvents/detergent, virus filtration, precipitation, chromatography, etc., in order to remove or inactivate viruses. Since acellular skin implants consist of three-dimensional protein structures, however, viruses are difficult to inactivate or remove from the acellular skin implants. Also, heat inactivation or treatment with low/high pH is of limited use because it is apt to denature the proteins of the skin implant. The chemical inactivation of viruses by solvent/detergent, commercialized and developed by the New York Blood Center in 1985, is found to effectively kill enveloped viruses, such as HIV, HBV, HCV, cytomegalovirus, etc., without any influence on protein activity. Thus, it can be used for the total inactivation of cytomegalovirus, which is observed to contaminate acellular skin implants at the highest frequency, with no negative influence on the proteins or collagens of the implant.

Korean Patent Publication No. 1994-1379 discloses living tissue equivalents comprising a hydrated collagen lattice contracted by a contractile agent, such as fibroblast cells. Korean Patent Laid-Open Publication No. 1993-700045 discloses composite living skin equivalents comprising an epidermal layer of cultured keratinocyte cells, a layer of highly purified, non-porous collagen and a dermal layer of cultured fibroblast cells in a porous, cross-linked collagen sponge. Korean Patent Laid-Open Publication No. 1992-336 describes a process of culturing keratinocytes in biocompatible perforated membranes. Douglas et al. used collagen gel as a scaffold for use in the preparation of artificial skin (In vitro 16:306-312, 1980). Cellulose or gelatin in a sponge was used as a scaffold for use in the preparation of artificial skin by Leighto et al., J. Natl Cancer Inst. 12:545-561, 1951; Cancer Res. 28:286-296, 1968. Keratinocytes and fibroblasts were cultured in a scaffold in the form of a membrane and non-woven mesh, prepared from hyaluronic acid derivatives (Valentian Zacchi et al., J. Biomed. Mater. Res. 40:187-194, 1998; Giampaolo Galassi et al., Biomaterials 21:2183-2191, 2000) (as described in lines 3-4, Korean Patent No. 10-0527623).

Korean Patent No. 10-0431659 (international filing date Jun. 15, 1998) discloses a wound covering material containing silk fibroin and silk sericin as main components and a process for producing the same.

Korean Patent No. 10-0315168 (filing date Jan. 28, 1999) discloses wound covering materials, prepared in the form of membranes by freeze-drying a silk fibroin protein solution, which are superior in biocompatibility, adhesion to a wound surface through partial fusion, moisture permeability, and skin regeneration.

Korean Patent No. 10-0386418 (applicant: Wellskin; filing date: Jul. 25, 2000) provides a skin equivalent prepared by culturing keratinocytes on a dermis equivalent constructed by combining an epidermis-free dermis layer with a fibroblast-containing collagen scaffold.

Korean Patent No. 10-0377784 (filing date, Jun. 22, 2000) provides an artificial skin prepared from mesenchymal cells of hair follicles, particularly beard follicles.

Korean Patent Application No. 10-2001-7014980 (international filing date Mar. 27, 2001) provides agents promoting the formation of artificial skin and agents stabilizing the skin basement membrane which contain a matrix metalloprotease inhibitor, optionally combined with a matrix protein production promoter; and a process for producing artificial skin by adding a matrix metalloprotease inhibitor, optionally combined with a matrix protein production promoter, to a medium for forming artificial skin.

Korean Patent No. 10-0527623 (filing date Jun. 1, 2002) provides a collagen scaffold for the preparation of artificial organs, prepared by culturing cells on a collagen scaffold and removing the cells while leaving the extracellular matrix (ECM) secreted from the cells. This patent teaches that the cells can be removed using a gamma irradiation process, a glycerol process, or an ethyloxide process. The gamma irradiation process may be implemented by freeze-drying the skin to separate the dermis from the epidermis, irradiating the dermis with a F-ray at a dose of 5,000 rad for 12 min, and storing the irradiated dermis in PBS for three weeks (N. C. Krejci et al., J. Invest Dermatol. 97:843-848, 1991). As for the glycerol process, cells are removed by treatment with glycerol in multiple steps, followed by washing with PBS (containing antibiotics) for 4 days (K. H. Chakrabarty, British Journal of Dermatology 141:811-823, 1999). In the ethylene oxide process, treatment with ethylene and then with 1 M NaCl for 8 hours is conducted before washing with PBS (containing antibiotics) for 6 weeks (K. H. Chakrabarty, British Journal of Dermatology 141:811-823, 1999). In an example of the patent, glycerol was used to remove cells.

Korean Patent Laid-Open No. 2001-0092985 (publication date Oct. 27, 2001) discloses an acellular dermal matrix and a process for preparing the same.

Korean Patent Application No. 2001-0005934 (filing date Feb. 7, 2001) describes the preparation of skin equivalents by isolating epithelial cells with trypsin and culturing them in an artificial structure.

U.S. Pat. No. 5,273,900 (filing date Sep. 12, 1991) provides a composite skin replacement consisting of an epidermal component in combination with a porous, resorbable, biosynthetic dermal membrane component which may be formed using collagen and muco-polysaccharide.

Like those mentioned above, conventional artificial skins containing collagen, gelatin or cellulose layers in the form of gel or a sponge generally do not retain sufficient tensile strength for suturing upon transplantation. Due to the low strength thereof, the skins are likely to curl or tear, giving rise to inconvenience in the operation. Further, the conventional artificial skins suffer from the disadvantage of being degraded by enzymes, such as collagenase, too quickly in consideration of the time period required for wound healing. In order to increase the strength of artificial skins, the crosslinking of collagen chemically with, e.g., glutaraldehyde or physically by UV irradiation has been studied. However, these chemical or physical techniques, although providing increased tensile strength for artificial skins, are apt to harden the bio-tissues or cause toxicity in cells or tissues in vivo. Another way to overcome the problems is to utilize a dermal scaffold, processed by removing cells from the skin of dead bodies, as a dermal implant or an insertion scaffold, like AlloDerm, commercially available from LifeCell, U.S.A. Another commercially available example is CCSR of Ortech, which uses a sponge collagen scaffold with fibroblasts and keratinocytes cultured therein. However, the dermal materials from dead bodies are disadvantageous in that they may be contaminated with various pathogens, such as the AIDS virus, and the supply thereof is limited (as described in Korea Pat. No. 10-0527623, supra).

Leading to the present invention, intensive and thorough research into dermal implants, conducted by the present inventors, resulted in the finding that the use of a solvent in combination with a detergent allows cells and viruses to be removed effectively and simultaneously.

### DISCLOSURE

### Technical Problem

It is therefore an object of the present invention to provide a method for producing an acellular implant for the human body by using a solvent in combination with a detergent to simultaneously remove cells and viruses from the implant.

### Technical Solution

In order to accomplish the above objects, the present invention provides a method for producing an acellular human body implant, comprising the use of tri(n-butyl) phosphate in combination with a detergent selected from a group consisting of deoxycholate, Tween 80, Triton X-100, sodium cholate and combinations thereof for conducting cell removal and viral inactivation simultaneously.

In the method, the acellular human body implant is a bone, a ligament, a muscle, or skin.

### DESCRIPTION OF DRAWINGS

FIG. 1 shows processes of preparing an acellular dermal implant in a stepwise manner, comprising separating the epidermis, which is apt to cause an immune rejection response (panels A and B), removing cells and inactivating viruses by treatment with solvent/detergent (panels C and D), and freeze drying the tissue and packaging it before use in patients (panels E and F).

FIG. 2 shows a fresh dermal tissue (A) and a dermal tissue treated with 2% deoxycholate (B).

FIG. 3 shows H & E-stained dermal tissues treated with 1% deoxycholate+0.3% TNBP (A) and 2% deoxycholate+0.3% TNBP (B) for various time periods.

FIG. 4 shows H & E-stained dermal tissues treated with 2% deoxycholate+0.1% TNBP (A) and 1% deoxycholate+0.1% TNBP (B) for various time periods.

FIG. 5 shows solvent/detergent-treated dermal grafts transplanted onto the subcutaneous layer (A) and their sizes (B).

FIG. 6 shows H & E-stained acellular dermal tissues sampled at various times after they were treated with 2% deoxycholate (A), 2% deoxycholate+0.2% TNBP (B), and 1% deoxycholate+0.1% TNBP (C).

FIG. 7 shows BHV viral PCR genes amplified by the use of primer-P1-F & primer-P1-R: 1(10³ TCID₅₀/ml); 2(10¹ TCID₅₀/ml), primer-P2-F & primer-P2-R: 3(10³ TCID₅₀/ml), 4(10¹ TCID₅₀/ml); primer-P3-F & primer-P3-R: 5(10³ TCID₅₀/ml), 6(10¹ TCID₅₀/ml); primer-P4-F & primer-P4-R: 7(10³ TCID₅₀/ml), 8(10¹ TCID₅₀/ml); and primer-P5-F & primer-P5-R: 9(103 TCID₅₀/ml), 10(10¹ TCID₅₀/ml), along with a 100 bp DNA ladder (M) and a negative control (NC).

FIG. 8 shows the sensitivity of PCR analysis for detecting the BHV virus:

M, 100 bp ladder; 8, 10⁸ TCID₅₀/ml; 7, 10⁷ TCID₅₀/ml; 6, 10⁶ TCID₅₀/ml; 5, 10⁵ TCID₅₀/ml; 4, 10⁴ TCID₅₀/ml; 3, 10³ TCID₅₀/ml; 2, 10² TCID₅₀/ml; 1, 10¹ TCID₅₀/ml; 0, 1 TCID₅₀/ml; NC, negative control.

FIG. 9 is a graph in which sequential dilutions of the BHV virus are plotted, showing the sensitivity of quantitative PCR analysis for detecting the BHV virus.

### BEST MODE

Hereinafter, embodiments of the present invention will be described in detail with reference to the attached drawings.

In accordance with an aspect thereof, the present invention pertains to a method for producing an acellular implant for the human body at low cost and high efficiency. The method features the use of a tri(n-butyl)phosphate (TNBP) solvent in combination with a detergent selected from among deoxycholic acid, Tween 80, Triton X-100, sodium cholate and a combination thereof.

In an embodiment of this aspect, an acellular implant for the human body can be produced by treating bio-materials of dead human bodies with tri(n-butyl)phosphate (TNBP) in combination with a detergent selected form among deoxycholic acid, Tween 80, Triton X-100, sodium cholate and a combination thereof to remove cells with the concomitant inactivation of viruses.

Examples of the implant for human bodies useful in the present invention include, but are not limited to, bones, muscles, ligaments, and skins. Simultaneous treatment as well as sequential treatment with the solvent and the detergent can allow the effective removal of cells and viruses at the same time.

Of the detergents, deoxycholate is preferably used in an amount from 0.1% to 5%. When the amount thereof is below 0.1%, dermal cells cannot be completely removed from the human donor tissue, which undergoes an immune rejection response upon transplantation. On the other hand, more than 5% of the detergent can remove dermal cells from the human donor tissue, but is apt to injure the extracellular matrix (collagen, elastin), a constituent of the dermis, giving rise to a decrease in the efficiency of engraftment.

The amount of the solvent tri(n-butyl)phosphate (TNBP) preferably falls within a range from 0.001% to 0.4%. Less than 0.001% of the solvent requires a long period of time for the treatment, which causes the human donor tissue to be damaged by other chemicals. On the other hand, when the solvent is used in an amount exceeding 0.4%, the extracellular matrix of the human donor tissue is negatively affected, resulting in difficulty in the angiogenesis and engraftment of the implant.

In accordance with the present invention, the treatment with deoxycholate and tri(n-butyl)phosphate (TNBP) is conducted preferably for a time period of 5-20 hours. If the treatment is finished within 5 hours, cells cannot be completely removed from the human donor tissue even though viruses are satisfactorily killed. On the other hand, a treatment period longer than 20 hours allows the complete removal of viruses and cells from the human donor tissue, but destroys the extracellular matrix, resulting in difficulty in the engraftment of the implant.

In another embodiment of the present invention, the method for producing an acellular human body implant comprises a dermis separating step, a cell removing step, a freeze protecting step, and a freeze-drying step, characterized in that deoxycholate is used as a solvent in combination with TNBP to remove cells and viruses simultaneously. The acelluar dermal implant of the present invention can be used as replacements for reconstructing the skin in patients with skin defect, such as burn, wound from traffic accidents, ulcers, etc. In addition, the acellular dermal implant is applicable to various loci of the human body, including the nasal septum as well as all skin layers in order to reconstruct injured skin, such as, e.g., reconstruction of injured dura mater, correction of depressed scars, correction of hemifacial microsomia, plastic reconstruction for lip enlargement, etc.

### Mode for Invention

A better understanding of the present invention may be obtained through the following examples which are set forth to illustrate, but are not to be construed as the limit of the present invention.

### Example 1

### Viral Clearance or Inactivation by Solvent/Detergent

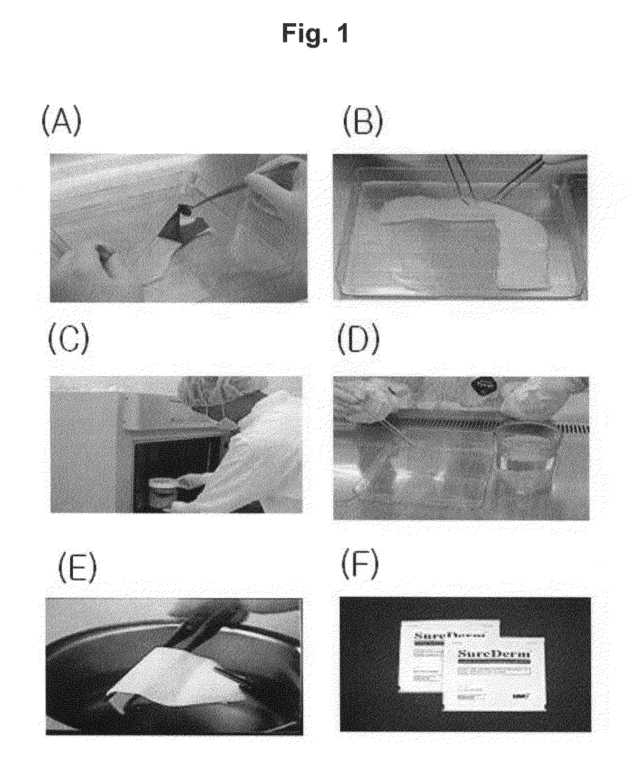

Acellular dermal implants are prepared from a human donor dermal tissue through a series of process steps including an epidermis separating step, a cell removing step, a freeze protecting step, and a freeze-drying step, as shown in FIG. 1. Of them, the cell removing step is configured to remove cells from the dermis of the human donor tissue with a low-molecular weight detergent. In the present invention, a detergent is used in combination with a solvent in order to remove cells as well as to inactivate viruses chemically.

To this end, deoxycholate was employed as a detergent and TNBP as a solvent useful in the cell removing process. Their concentrations were determined so as to be able to remove cells and concomitantly kill enveloped viruses, without any negative influence on the collagen matrix of the acellular dermal implant. Deoxycholate was used in an amount of 1% or 2%, while the amount of TNBP was 0.3%, which is sufficient to inactivate viruses. Thus, human donor dermal tissues were treated with combinations of 1% deoxycholate+0.3% TNBP and 2% deoxycholate+0.3% TNBP for 5 h, 10 h, 15 h, 20 h and 24 h, and the results are given in FIG. 3.

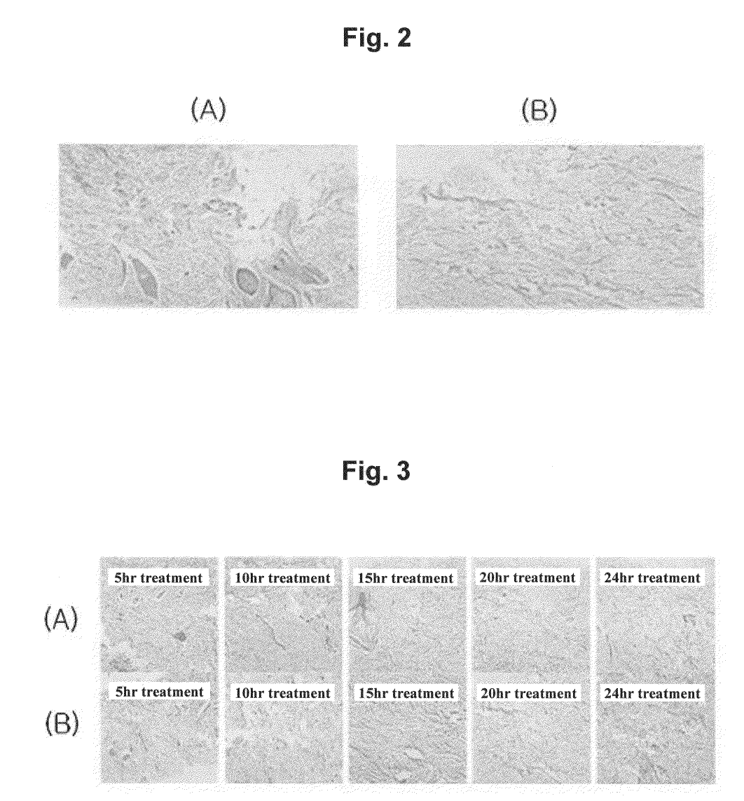

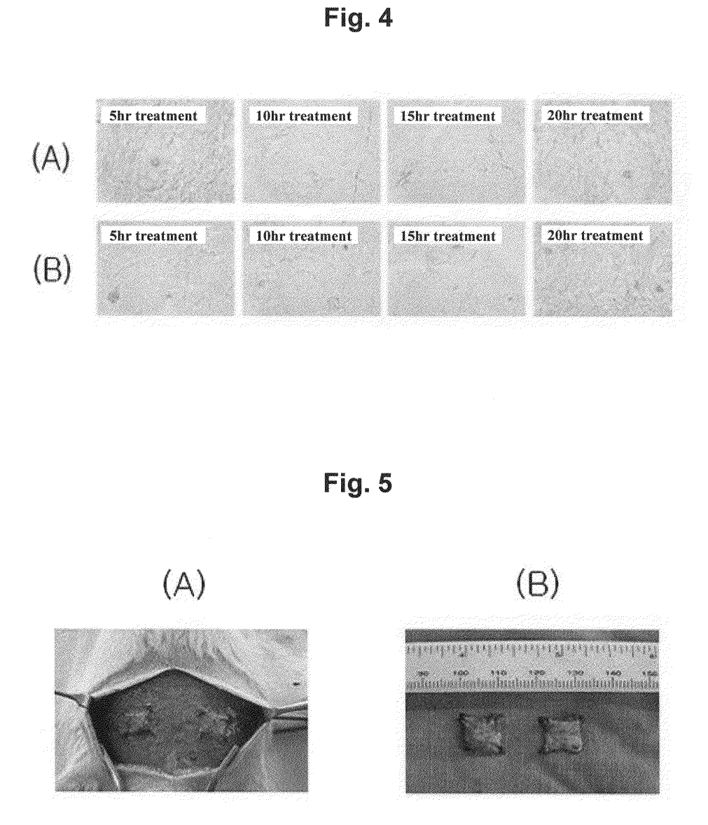

After being treated with 1% deoxycholate+0.3% TNBP or 2% deoxycholate+0.3% TNBP, the dermal tissues were subjected to histological assay for various time periods to determine whether cells were removed from the dermis of the tissue and the micro structure of the collagen matrix underwent a change. In contrast to the dermal tissue which was treated using only a solvent according to a conventional method (FIG. 2), the dermal tissues were found to become free of cells after treatment with either 1% deoxycholate+0.3% TNBP (panel A of FIG. 3) or 2% deoxycholate+0.3% TNBP (panel B of FIG. 3), with no observations of the effect of TNBP on the cell removal. With the increase in the time period of treatment with 1% deoxycholate+0.3% TNBP or 2% deoxycholate+0.3% TNBP, the collagen matrix was observed to undergo deformation and change in micro structure (FIG. 3). When account was taken of the fact that no change in the micro structure of the dermis took place even after treatment with 2% deoxycholate for 24 hours, TNBP was believed to influence the collagen structure of the dermis. TNBP was applied in various amounts from 0.3% to 0.1% to human donor dermal tissues in order to determine a concentration that was effective at removing cells with a minimum of injury to the collagen matrix of the dermis. In all tissues treated with 1% deoxycholate+0.1% TNBP or 2% deoxycholate+0.1% TNBP, it was observed that cells were removed without injury on the collagen matrix over time (FIG. 4).

### Example 2

### Assay for Graft Product of Solvent/Detergent Method for Biocompatibility

1. Histological Observation of Acellular Dermal Implant

In order to examine whether the acellular dermal implants treated with various concentrations of solvent/detergent underwent a change in the micro structure of the matrix thereof over time, they were subject to histological assay. In this regard, the tissues were sectioned and stained with H & E (hematoxylin and eosin) to visualize the nucleus and the cytoplasm distinctively under a microscope.

2. Assay for Safety of Acellular Dermal Implant

In order to examine whether the solvent/detergent solution remaining in the acellular dermal implants had toxic effects on cells of the patient, the exudates from the grafts were assayed for cell lysis (cell death) and inhibition of cell growth according to U.S. Phamacopoeia <87> (biological reactivity tests in vitro, for physical/chemical/biological safety of a sample). The acellular dermal implants obtained after treatment with the solvent/detergent were powdered and mixed with 1 ml of distilled water for every 300 mg of the powder. To 4 g of the mixed sample was added 20 ml of physiological saline, followed by extraction at 37° C. for 72 hours. Tests were conducted within 24 hours after the completion of the extraction. The extract was mixed at a volume ratio of 1:2 with a culture medium (MEM). L-929 cells (fibroblasts), which are usually used in inhibition tests because of their high susceptibility to chemicals, were plated at a density of 10⁵ cells per ml before culturing. The L-929 cells were supplemented with a test exudate, a negative solution and a positive solution, as shown in Table 1, below, and incubated for 48 hours before microscopic observation for cell morphology, pores, detachment, lysis, etc. This experiment was repeated three times while cytotoxicity was evaluated according to the qualitative method of USP <87>. The results are summarized in Table 2, below.

TABLE 1
Samples for Cytotoxicity Assay

| | Acellular dermal implants treated with |
|---|---|
| Sample | solvent/detergent |
| Negative | Physiological saline |
| Control | |
| Positive | DMSO |
| Control | |
| Exudate | From acellular dermal implants treated with |
| | solvent/detergent |

TABLE 2
Evaluation Grades of Cytotoxicity

| Grades | Reactivity | Culture Conditions |
|---|---|---|
| 0 | None | Granules within separated cells: no cll |
| | | lysis |
| 1 | Slight | As much as 29% of the cells become round |
| | | and loosened accompanied by the |
| | | disappearance of granules: cells lysates |
| | | found often |
| 2 | Mild | As much as 50% of the cells becomes |
| | | round with the disappearance of |
| | | granules: significant cell lysates and |
| | | no voids between cells |
| 3 | Moderate | As much as 70% of the cells become round |
| | | or lyze |
| 4 | Serious | Almost all cells disrupted |

3. Assay for Histocompatibility of Acellular Dermal Implant

The acellular dermal implants treated with the solvent/detergent were transplanted onto the subcutaneous layer in rats and assayed for histocompatibility with time. To this end, first, the implants treated with the solvent/detergent were cut to a size of 1×1 cm and hydrated in saline just before transplantation. 15 Sprague-Dawley rats, each weighing around 200 g, were used for this assay. After being anaesthetized with ketamine and xylazine, the experimental animals were shaved around the backbone and the hydrated acellular dermal implants were transplanted onto the subcutaneous layer. On Week 2, 4, 6, 8 and 10 after the transplantation, tissues were taken from the experimental animals, fixed in 10% formalin for 24 hours, embedded in paraffin, sliced to a thickness of 5 μm and stained with H & E before observation under an optical microscope.

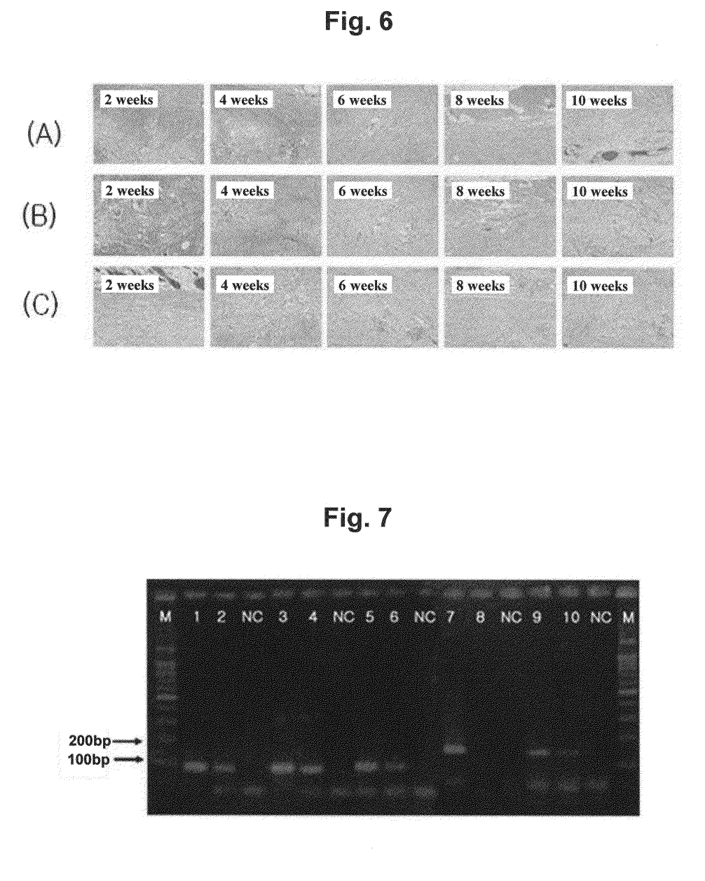

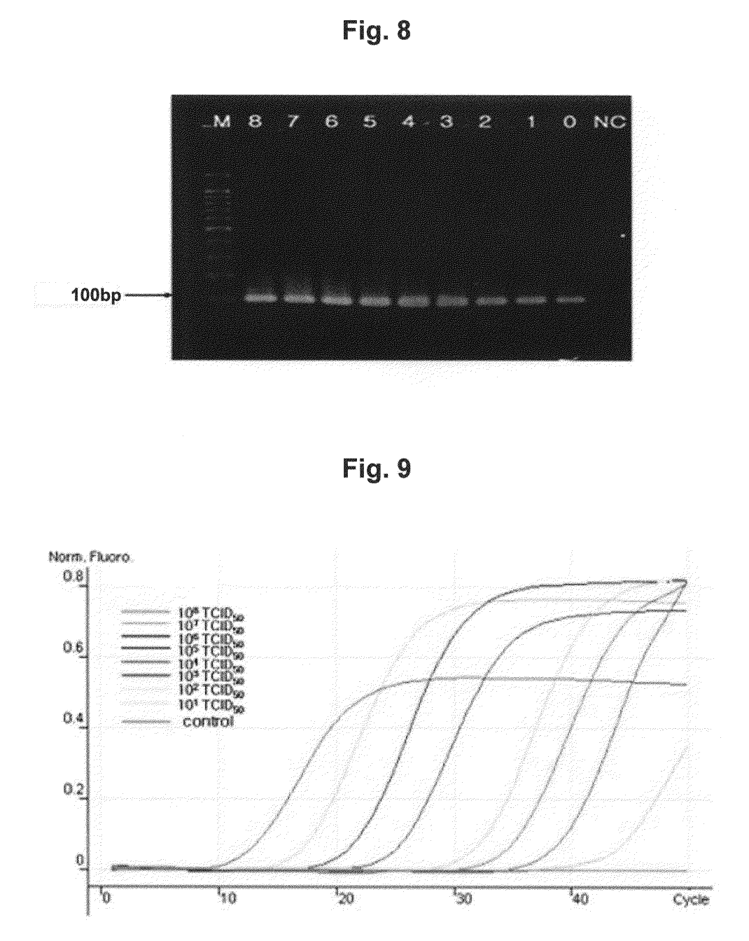

Acellular dermal tissues obtained by treatment with 2% deoxycholate, 2% deoxycholate+0.2% TNBP, and 1% deoxycholate+0.1% TNBP were transplanted onto the subcutaneous layer of the experimental animals (FIG. 5) and examined for biocompatibility 2, 4, 6, 8, and 10 weeks after the transplantation (FIG. 6). As seen in FIG. 6, a significant number of inflammatory cells, such as lymphocytes, were observed around all of the grafts treated with 2% deoxycholate, 2% deoxycholate+0.1% TNBP, and 1% deoxycholate+0.1% TNBP in Week 2 and 4. With the passage of time, however, the inflammatory cells were observed to decrease in number with the infiltration of some vascular cells into the graft. Particularly, the acellular dermal graft treated with 1% deoxycholate+0.1% TNBP showed excellent histological properties free of inflammatory responses and calcification and were observed to be low in absorption over time. In the grafts treated with 2% deoxycholate+0.1% TNBP and 1% deoxycholate+0.1% TNBP, no significant inflammatory responses sufficient to have an influence on the human body were observed.

### Example 3

### Assay for Safety of Acellular Dermal Implants Treated with Solvent/Detergent

In order to examine whether the solvent/detergent solution remaining in the acellular dermal implants had toxic effects on cells of the patient, the exudates from the grafts were assayed for cytotoxicity. Evaluation was made according to the grade mentioned above. Both the exudates from the grafts treated with 2% deoxycholate alone and 1% deoxycholate+0.1% TNBP showed neither necrosis nor cell lysis while cells underwent a slight morphological change when the exudate from the graft treated with 2% deoxycholate+0.1% TNBP was used. Thus, both the exudates from the graft treated with 2% deoxycholate alone and 1% deoxycholate+0.1% TNBP were determined to be grade zero, and the exudates from the graft treated with 2% deoxycholate+0.1% TNBP was determined to be grade 1 (Table 3).

TABLE 3
Exudate Grades

| | Exudates | | | | | | Negative | | | Positive | | |
|---|---|---|---|---|---|---|---|---|---|---|---|---|
| Time | A | B | C | Medium | | | Control | | | Control | | |
| 24 hours | 0 | 1 | 0 | 0 | 0 | 0 | 0 | 0 | 0 | 4 | 4 | 4 |

A: from the graft treated with 2% deoxycholate alone
B: from the graft treated with 2% deoxycholate + 0.1% TNBP
C: from the graft treated with 1% deoxycholate + 0.1% TNBP

### Example 4

### Clearance and Inactivation of Pathogens (Cytomegalovirus) by Treatment with Solvent/Detergent

In assay for the clearance and inactivation of cytomegalovirus, bovine herpes virus (BHV) was employed as a model virus of cytomegalovirus.

1. Culture of BHV

Madin-Darby bovine kidney (MDBK) cells (ATCC CRL-22) were used as a host for BHV(ATCC VR 188). MDBK cells were cultured in Dulbecco's Minimum Essential Medium (DMEM, Gibco BRL) supplemented with 10% fetal bovine serum (FBS: Gibco BRL, Gaithersburg, USA). A MDBK monolayer grown in a T-175 flask was infected with BHV and periodically monitored for CPE (cytopathic effect). When CPE was apparently visualized, the cell culture was centrifuged at 400×g for 7 min. The cell pellet was resuspended while the supernatant was collected separately. The pellet suspension was subjected to two cycles of freezing and thawing to disrupt the cells, followed by centrifugation at 400×g for 7 min. The supernatants thus obtained were pooled and filtered through a 0.45 μm filter, and the filtrate was aliquoted before storage at −70° C.

2. BHV Recovery Test

An assay for viral inactivation was conducted by spiking viruses into skin tissues, drying the tissues naturally and recovering the viruses. In order to optimize the condition for recovering the virus and determine the recovery rate, first, BHV was spiked into samples and recovered using a PBS buffer and a cell culture medium.

Also, recovery rates were measured in the presence of 0.1% Triton x100 or 0.1% Tween 80. The results are summarized in Table 4, below. As seen in Table 4, the recovery rates fell within the range from 15 to 26%, which was relatively uniform for all of the reagents. Due to the high likelihood of detergents such as Triton X100 and Tween 80 destroying the virus, the spiked BHV was recovered using PBS.

TABLE 4
Recovery Rates of Virus

| Media | Recovery Rates (%) |
|---|---|
| PBS | 15.4 |
| PBS + 0.1% Triton X100 | 24 |
| PBS + 0.1% Tween 80 | 26 |
| Culture medium | 18.8 |
| Culture medium + 0.1% Triton X100 | 15.4 |
| Culture medium + 0.1% Tween 80 | 24 |

3. Assay for BHV Inactivation by Treatment with Solvent/Detergent

(1) BHV Inactivation in Solvent/Detergent

To 54 ml of the solvent/detergent solution was added 6 ml of BH. Immediately, 6 ml of the resulting sample was diluted 64-fold with a culture medium, which is a dilution factor sufficient to prevent viral cytotoxicity and interference, followed by titration. While the remainder was incubated at 20° C., 6 ml of the solution was sampled at intervals of 5 min, 30 min, 60 min and 120 min. As soon as each sample was diluted 64-fold, it was measured for titer. The results are summarized in Table 5, below. As seen in the data of Table 5, almost all viruses were killed within 5 min after BHV was spiked in the solvent/detergent solution. Quantitative analysis showed that no living viruses were detected 30 min after the BHV spiking in the solvent/detergent. After treatment with the solvent/detergent solution, the inactivation rate was ≧log 4.32. From these results, it was understood that BHV was completely inactivated by the solvent/detergent solution.

TABLE 5
BHV Inactivation in Solvent/Detergent

| | | Overall BHV Titer |
|---|---|---|
| Experiment # | Samples | (Log₁₀ TCID₅₀) |
| 1 | Spiked initiate | 7.84 |
| | 5 min after treatment with | 3.58 |
| | solvent/detergent | |
| | 30 min after treatment with | ND¹ (3.47)² |
| | solvent/detergent | |
| | 60 min after treatment with | ND¹ (3.47)² |
| | solvent/detergent | |
| | 120 min after treatment with | ND¹ (3.47)² |
| | solvent/detergent | |
| 2 | Spiked initiate | 7.74 |
| | 5 min after treatment with | 3.68 |
| | solvent/detergent | |
| | 30 min after treatment with | ND¹ (3.47)² |
| | solvent/detergent | |
| | 60 min after treatment with | ND¹ (3.47)² |
| | solvent/detergent | |
| | 120 min after treatment with | ND¹ (3.47)² |
| | solvent/detergent | |

¹No BHV infection found
²calculated using the theoretical minimal observations with 90% reliability.

(2) BHV Inactivation by Treatment with Solvent/Detergent in the Preparation Process

Under a condition for scaling down the preparation process of the present invention, BHV inactivation by treatment with the solvent/detergent in the preparation process was evaluated. A dermal tissue from which the epidermis was removed was cut to a size of 4×5 cm, spiked with 4 ml of BHV, and dried naturally on a clean bench. While being treated with the solvent/detergent in the same manner as in the preparation process, the tissue samples were recovered at intervals of 0, 5, 30, 60, and 120 min and washed four times with a washing solution to remove the solvent/detergent. The titers of the samples were measured as soon as they were recovered. The results are summarized in Table 6. As seen in the data of Table 6, almost all viruses were killed within 5 min after BHV was spiked in the solvent/detergent solution. Quantitative analysis showed that no living viruses were detected 30 min after the BHV spiking in the solvent/detergent. After treatment with the solvent/detergent solution, the inactivation rate was ≧log 6.43. From these results, it was understood that BHV was completely inactivated by the treatment process according to the present invention.

TABLE 6
BHV Inactivation by Treatment with Solvent/Detergent

| | | Overall BHV Titer |
|---|---|---|
| Experiment # | Samples | (Log₁₀ TCID₅₀) |
| 1 | Spiked initiate | 7.96 |
| | 5 min after treatment with | 1.59 |
| | solvent/detergent | |
| | 30 min after treatment with | ND¹ (1.48)² |
| | solvent/detergent | |
| | 60 min after treatment with | ND¹ (1.48)² |
| | solvent/detergent | |
| | 120 min after treatment with | ND¹ (1.48)² |
| | solvent/detergent | |
| 2 | Spiked initiate | 7.86 |
| | 5 min after treatment with | 1.69 |
| | solvent/detergent | |
| | 30 min after treatment with | ND¹ (1.48)² |
| | solvent/detergent | |
| | 60 min after treatment with | ND¹ (1.48)² |
| | solvent/detergent | |
| | 120 min after treatment with | ND¹ (1.48)² |
| | solvent/detergent | |

¹No BHV infection found
²calculated using the theoretical minimal observations with 90% reliability.

### INDUSTRIAL APPLICABILITY

As described above, the present invention provides acellular dermal implants, which meet the requirements stipulated by the regulations of the ISO and the FDA with regard to transplants for the human body, and are superior to conventional ones in terms of safety. Also, a method is provided for producing the acellular dermal implants, featuring chemical viral inactivation by treatment with a solvent/detergent solution. By the method, enveloped viruses, such as cytomegalovirus, HIV, HBV, HCV, etc., can be effectively killed without negative influencing the acellular dermal layer.

According to the present invention, the use of the detergent deoxycholate in combination with 0.1% of tri(n-butyl)phosphate (TNBP) makes it possible to conduct the removal of cells from the dermal layer and the inactivation of the virus. The acellular dermal implants produced according to the present invention are found to be safe and highly biocompatible as measured by ex vivo animal assays and in vitro cytotoxicity assays. Therefore, the present invention can produce implants for human body at low cost with high efficiency.

1. A method for producing an acellular human body implant, comprising the use of tri(n-butyl)phosphate in combination with a detergent selected from a group consisting of deoxycholate, Tween 80, Triton X-100, sodium cholate and combinations thereof in conducting cell removal and viral inactivation simultaneously.

2. The method according to claim 1, wherein the acellular human body implant is a bone, a ligament, a muscle, or skin.

3. The method according to claim 1, wherein the method comprises an epidermis separating step, a cell removing step, a freeze protecting step, and a freeze-drying step and is characterized by the use of tri(n-butyl)phosphate in combination with deoxycholate.

4-6. (canceled)

7. An acellular human body implant, produced using the method of claim 1.

8. A wound healing agent, comprising the acellular human body implant of claim 7.

9. The method according to claim 1, wherein deoxycholate is used in a concentration ranging from 0.1% to 5%.

10. The method according to claim 2, wherein deoxycholate is used in a concentration ranging from 0.1% to 5%.

11. The method according to claim 1, wherein tri(n-butyl)phosphate is used in a concentration ranging from 0.001% to 0.4%.

12. The method according to claim 2, wherein tri(n-butyl)phosphate is used in a concentration ranging from 0.001% to 0.4%.

13. The method according to claim 1, wherein the use of tri(n-butyl)phosphate in combination with deoxycholate is carried out by applying tri(n-butyl)phosphate and deoxycholate to a bio tissue for 5 to 20 hours.

14. The method according to claim 2, wherein the use of tri(n-butyl)phosphate in combination with deoxycholate is carried out by applying tri(n-butyl)phosphate and deoxycholate to a bio tissue for 5 to 20 hours.
